SEQUENCE LISTING (7 sequences)

SEQ ID NO: 1 - DNA, 27 bp, Artificial Sequence
  Oligonucleotide
aacggatccatgttattcaagtctttc

SEQ ID NO: 2 - DNA, 26 bp, Artificial Sequence
  Oligonucleotide
gggctgcaggtgcttagttagagtaa

SEQ ID NO: 3 - DNA, 27 bp, Artificial Sequence
  Oligonucleotide
atagagctcgtgcgaaattcgtctaat

SEQ ID NO: 4 - DNA, 30 bp, Artificial Sequence
  Oligonucleotide
cttggtacccgatgacttctaactctttct

SEQ ID NO: 5 - DNA, 24 bp, Artificial Sequence
  Oligonucleotide
gctgtagtgccattgattcgtaac

SEQ ID NO: 6 - DNA, 23 bp, Artificial Sequence
  Oligonucleotide
ctggatctcttcctttaccaaac

SEQ ID NO: 7 - DNA, 26 bp, Artificial Sequence
  Oligonucleotide
catggaggcctcatggtttggatttc

(19) United States
(12) Patent Application Publication
(10) Pub. No.: US 2009/0081196 A1
(43) Pub. Date: Mar. 26, 2009

(54) Medicaments for fungal infections

(75) Inventor: Antonio Cassone, Rome (IT)

Correspondence Address: QUINE INTELLECTUAL PROPERTY LAW GROUP, P.C., P O BOX 458, ALAMEDA, CA, 94501, US

(73) Assignee: Istituto Superiore di Sanita, Roma (IT)

(21) Appl. No.: 12/070,335

(22) Filed: Feb. 15, 2008

Related U.S. Application Data
(60) Provisional application No. 60/901,853, filed on Feb. 16, 2007.

(51) Int. Cl. A61K 39/00; A61K 39/395; C12N 5/02; A01N 3/00; C07K 16/00

(52) U.S. Cl. 424/130.1; 435/375; 435/1.1; 530/387.1; 530/387.3; 424/274.1

(57) ABSTRACT

The protein CAMP65p of C. albicans has been found to be play a significant role in both adhesion and the production of hyphae, which are important factors in both virulence and resistance to clearing. This invention provides antibodies to CAMP65p useful for administration to patients for prophylaxis and treatment of candidal infections.

### CROSS-REFERENCE TO RELATED APPLICATIONS

This application claims priority to and benefit of a prior U.S. Provisional Application No. 60/901,853, Medicaments for Fungal Infections, by Antonio Cassone, filed Feb. 16, 2007. The full disclosure of the prior application is incorporated herein by reference.

### FIELD OF THE INVENTION

The present invention is in the field of medicaments useful for treatment of fungal infections. Particularly, the medicaments include vaccines or antibodies directed to Candida albicans mannoprotein 65 (CAMP65).

### BACKGROUND OF THE INVENTION

Yeasts belonging to genus Candida comprise a number of species that are major pathogens for immunocompromised hosts. For instance, Candida albicans ranks fourth among the most common agents of bloodstream infections in the hospitalized, immunocompromised patient (Pfaller et al., 1998). The same fungus is a very frequent cause of mucosal, in particular vaginal, infections in otherwise normal subjects (Fidel and Sobel, 1996). The fungal factors which express the pathogenic potential of Candida spp. have been extensively investigated, but no definite factor has emerged as being solely responsible. Rather, a team of aggressive traits appears to cooperate, to a variable extent, in the different forms of candidiasis (Calderone and Fonzi, 2001; Cutler, 1991).

In the context of Candida infections, cell wall mannoproteins (MPs) have been investigated as significant players in the host-parasite relationship. Being surface-located and secreted to the external milieu, MPs are thought to be involved in cell-cell recognition and response to stress factors. Particularly, MPs warrant consideration as potential aggressive factors and as a major target of host immune responses. This dual role can be best exemplified by the recognized capacity of some MPs to favour the adhesion of the fungus to host cell surface, i.e. the essential first step in pathogenicity and disease transmission, as well as by their nature of being critical targets of both humoral and cell-mediated immune (CMI) responses by the host (Cassone, 1989). Related to this, MPs are quite versatile, bi-functional molecules with double chemical entities, the saccharide and the protein, both of which are clearly involved in host-Candida relationship, since both can work as adhesins and both elicit antibodies during colonisation and infection (Mansour and Levitz, 2003). Antibodies against some MPs have been shown to be protective (Yuan et al., 1995), though other anti-MP antibodies have been shown to actually compete with protective antibodies (Bromuro et al., 2002). On the other hand, the CMI response, which is usually directed against the protein moiety of MPs, is promptly detectable in almost all healthy subjects, and is popularly considered to play a critical role in anti-Candida defense (Romani, 1997).

Camp65 is a 65 kilodalton mannoprotein that has been the subject of some research (Cassone et al., 1998). It is present in the cell wall of both yeast and hyphal forms of C. albicans (Bromuro et al., 1994) and has been shown to be a main target of cell-mediated immune response against C. albicans (Nisini et al., 2001; Gomez et al., 2000; Torosantucci et al., 1991). There are some indications that both this response and antibodies collaborate in protecting the animal from systemic or mucosal C. albicans challenge (Mencacci et al., 1994, De Bernardis, 2006 #94). The CAMP65 gene was recently cloned and a recombinant, 46 kilodalton, 6-histidine tagged protein (Camp65p) expressed, which was as extensively recognized by the human lymphocytes in vitro as the native Camp65 (Nisini et al., 2001; La Valle et al., 2000). It possesses an RGD signature putatively involved in adhesion mechanisms (Calderone et al., 2000; Gale et al., 1998).

However, the biological and virulence properties, if any, of this protein have never been formally investigated. Candida infections remain a substantial and difficult to treat threat, especially in immune-compromised individuals. Moreover, options for anti-fungal treatments are quite limited and resistance is becoming more common.

In view of the above, a need exists for a treatments to prevent Candida infections or to help resolve active infections. A need remains for effective treatments against Candida infections on surfaces. The present invention provides these and other features that will be apparent upon review of the following.

### SUMMARY OF THE INVENTION

We have now surprisingly found that CAMP65 is necessary both for adhesion, thereby confirming earlier research, but also for hyphal production. Hyphae are necessary for the fungus to be able to block clearance, and we have also found that the adhesion engendered by CAMP65 is associated with virulence, and we have discovered that it is possible to block both effects, thereby both weakening the ability of C. albicans to infect and reducing the resistance of the fungus to clearance.

Thus, in a first aspect, the present invention provides the use of an antibody-like molecule specific for CAMP65p of Candida albicans in the manufacture of a medicament for the treatment or prophylaxis of a fungal infection. The present invention also provides medicaments comprising such antibody-like molecules.

The infecting fungus will typically be Candida and, more especially, C. albicans, but may be any related fungus which expresses the CAMP65 protein, or such a closely related protein that anti-CAMP65 antibodies bind thereto.

In a particularly preferred embodiment, the medicament of the invention is for the treatment or prophylaxis of a C. albicans infection.

The medicaments of the invention are useful in both early stage and late stage infection, as well as prophylaxis, as blocking CAMP65 has the dual effect of blocking both adhesion and hyphal formation, so that it acts as a preventative measure for adhesion and persistence, helping to clear established infection, and also preventing hyphal formation and adhesion of the hyphae, which helps to prevent establishment of the infection and eases subsequent clearance of the infecting fungus.

In a preferred embodiment, the invention comprises a composition including an antibody specific for CAMP65 or CAMP65p in a formulation for topical administration. For example, the composition can be CAMP65 compounded in a formulation such as a suppository, cream, paste, ointment, gels, or the like, e.g., for administration to a tissue surface of a patient. In certain embodiments, the formulation does not consist of an aqueous solution, e.g., suitable for parenteral administration.

In a preferred embodiment, the invention can be a method of preventing adhesion of a fungus to a surface, e.g., by contacting a Candida sp. with an antibody-like molecule specific for CAMP65 in an amount sufficient to reduce adhesion of the Candida sp. to the surface as compared to adhesion of the Candida sp. without the contacting with the antibody-like molecule. As discussed below, an “antibody-like molecule” can be, e.g., an antibody, antibody fragment or peptide having sequences of a CAMP65 antibody variable region. In a more preferred embodiment, the Candida sp. is Candida albicans. In many embodiments, the surface upon which Candida adherence is inhibited is, e.g., a polymer surface, a cell surface, a tissue surface, an organ surface, an external surface of an animal, and/or the like.

The medicaments of the invention, whilst being able to be prepared as injectables, will often be prepared as external applications. Suitable external formulations include pessaries, suppositories, flushes, creams, pastes, ointments, and gels, and such formulations will typically be made up with suitable excipients, such as vehicles, bulking agents, gelling agents and sterilants.

The medicaments of the present invention may also comprise a further active ingredient, such as a further anti-fungal agent, for example.

Preferred medicaments of the present invention comprise at least one further antibody-like molecule to another determinant of the fungus to be treated. The preferred fungus is C. albicans.

In an alternative aspect, the present invention provides a vaccine for candidiasis comprising all or part of CAMP65 in an antigenic form, optionally together with an adjuvant and/or one or more further antigenic determinants from the fungus to be treated.

The nature of the antigen-presenting molecule is not important, provided that it serves to stimulate an immunogenic response, and particularly an antibody response, against the CAMP65 molecule.

Such vaccines will typically be formulated as liquids for injection. The injection type is not important, but will typically be intravenous.

### DEFINITIONS

Unless otherwise defined herein or below in the remainder of the specification, all technical and scientific terms used herein have meanings commonly understood by those of ordinary skill in the art to which the present invention belongs.

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a component” can include a combination of two or more components; reference to “feed” can include mixtures of feed, and the like.

Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

The term “antibody-like molecules” is used to indicate molecules which have binding specificity for a given substance. As used hereinabove, the specificity is for CAMP65, and the molecules can be human or animal antibodies, humanized antibodies, antibody fragments, such as Fab fragments, and engineered fragments of antibodies, as well as substances incorporating a domain or region corresponding to that of a variable region of an antibody specific for CAMP65.

### BRIEF DESCRIPTION OF THE DRAWINGS

FIG. 1A to 1D panels show gels and micrographs associated with targeted disruption of the CAMP65 gene. 1A panels show Southern blot analysis: total digested chromosomal DNA was subjected to agarose gel electrophoresis and transferred to nylon membrane. The membrane was probed with a biotin-labeled CAMP65, RPS1 and URA3 DNA fragments, hybridized and detected as detailed in Experimental Procedures. 1B panels show Northern blot analysis: total RNA was extracted from exponentially growing cells cultured for 24 h at 37° C. in modified Lee's medium. Equal amounts of RNA were subjected to denaturant electrophoresis, blotted to Hybond-N membrane and hybridized with CAMP65, URA3 and rDNA probes, as specified in Experimental Procedures. 1C panels show Western blot analysis: concentrated supernatants of exponentially growing cells cultured for 24 h at 37° C. in modified Lee's medium were used as described in Experimental Procedures. Equal amounts of secreted proteins were subjected to immuno-blot analysis, reacted with 4C8 anti-Camp 65p monoclonal antibody, anti-mannan mAb AF1 and mouse anti-Camp65p polyclonal antiserum and detected as specified in Experimental Procedures. Electrophoretic mobility of purified, recombinant Camp65p (from pRLV130) (La Valle et al., 2000) and low range pre-stained marker (Biorad) is shown. 1D panels show Immunofluorescence analysis wherein wild type and CAMP65 mutants cells were grown in Lee's medium at 37° C. for 2 h and reacted with the anti-Camp65p mAb 4C8 and FITC-conjugate goat anti-mouse IgG as described in details in Experimental procedures.

FIGS. 2A and 2B show that CAMP65 is required for hyphal morphogenesis and germ-tube formation. 2A panels show colony morphology of CAMP65 mutant strains on solid media. Mutant and wild type strains were plated in Spider and modified Lee's medium supplemented with uridine and incubated at 37° C. Peripheral hyphae morphologies after 3 days of growth of each strain on Lee's and Spider media are shown. The magnification bar corresponds to 0.8 mm. The chart at 2B shows germ tube formation rates for various CAMP65 mutant strains. Wild type and mutant strains were inoculated in uridine-supplemented M199 medium, incubated at 37° C. for 2 h and the percentage of germ tubes counted under the light optical microscope. Values are mean (±SD) of triplicate independent samples. * denotes a statistically significant difference (P<0.05, Student's t test, two tailed;) as compared to the wt strain, while ** denotes a statistically significant (P as above) difference as compared to het strains. NS denotes non significant as compared to the wt strain.

FIGS. 3A and 3B charts show that Camp65p is required for adherence to the plastic. FIG. 3A, shows the relative adherence properties of various CAMP65 mutant strains. Each strain was inoculated in modified Lee's medium and incubated at 37° C. for 3 h in polystyrene wells. After washing, plastic-adherent cells were incubated in solid media for 24 h, colonies were counted and the results expressed as percentage of the inoculated cells. Values are mean (±SD) of triplicate independent assays. *denotes a statistically significant difference (P<0.05, Student's t test, two tailed) as compared to the wt strain; **denotes a statistically significant difference (P as above) as compared to both het and rev strains. FIG. 3B, shows inhibition of C. albicans adherence to plastic resulting from exposure to antibodies against Camp65p along with control data. Wild type C. albicans cells were incubated with the indicated IgG-rich fractions, purified from immune and non-immune mouse sera as specified in the Experimental Procedures. Values are mean (±SD) of triplicate independent determinations. * denotes a statistically significant difference (P<0.05, Student's t test, two tailed) as compared to the treatment with the non-immune serum, while ** denotes a statistically significant difference (P as above) as compared to the anti-Scw immune serum. NS, non significant.

FIGS. 4A and 4B show experimental pathogenicity of CAMP65 mutants in mouse bloodstream infections. FIG. 4A shows Kaplan-Meyer curves of mouse survival following experimentally induced candidiasis. The virulence of mutant strains was compared to that of the wild type strain in an intravenous murine model of haematogenously disseminated candidiasis. CD2F1 mice (10 mice per group) were infected via tail vein with 5×10⁵ yeast-form cells of C. albicans and animal survival was monitored for 30 days. The median survival times (MST, days) of the null mutant were significantly different (P<0.01, Mann-Withney U test, two tailed) between the null mutant and both the wt strain and the heterozygous strains. There was also a significant difference (P<0.05, as above) in the MST between the null mutant and the heterozygous strains. FIG. 4B shows kidney sections of infected mice. Kidney of mice challenged with wild type and mutant strains were removed from euthanized mice, 3 days post-challenge, fixed, dehydrated and stained with Grocott as described in the Experimental Procedures. The magnification bar corresponds to 0.6 mm and 30μ for the upper and lower panels, respectively.

FIGS. 5A and 5B show experimental pathogenicity of C. albicans strains in a mucosal (rat vaginal) infection model. FIG. 5A shows CFU counts in the vaginal fluid from rats intravaginally infected with each strain and monitored up to 28 days. *denotes a statistically significant difference (P<0.05, Mann-Withney U test, two tailed) between the CFUs of the null mutant and those of both the parent and revertant strains. For other technical details, see Experimental Procedures. FIG. 5B shows cytological analysis of vaginal scraping taken from infected rats. Vaginal scrapings were taken 2 days after the challenge from rats infected with wild type, homozygous and revertant cells. Slides were stained with periodic acid-Schiff-van Gieson stain and observed under a light microscope. Representative fields were randomly selected and micro-photographed as described in the Experimental procedures. The magnification bar corresponds to 60 μm.

### DETAILED DESCRIPTION

We constructed a series of CAMP65 knockout mutants that were investigated for their phenotype, growth characteristics, morphogenesis, adherence to plastic and pathogenicity. The CAMP65 knock-out mutants that were constructed included null camp65/camp65 and CAMP65/camp65 heterozygous strains. The null strains were viable and grew well in the yeast form but they were severely affected in hyphal morphogenesis both in vitro and in vivo. Hyphae formation was restored in revertant strains. The null mutants also lost adherence to plastic, and this was in keeping with the strong inhibition of fungal cell adherence to plastic exerted by anti-Camp65p antibodies.

The null mutants were also significantly less virulent than the parental strains, and this loss of virulence was observed both in systemic and in mucosal C. albicans infection models. Nonetheless, the virulence in both infectious models was regained by the CAMP65 revertants. Thus, CAMP65 of C. albicans encodes a β-glucanase adhesin, which has a dual role (hyphal cell wall construction and virulence), accounting for the particular relevance of host immune response against this mannoprotein.

Camp65p is a 65 kilodalton mannoprotein of C. albicans previously discovered and characterized as a main antigen target of anti-Candida immune response in humans (Gomez et al., 1996; Bromuro et al., 1994; Torosantucci et al., 1991). The CAMP65 gene encodes a 48 kDa protein with one N- and multiple O-glycosylation sites (La Valle et al., 2000) which belongs to a family of beta-glucanase or trans-glycosidase enzymes known to play a critical function in maintaining the integrity of the cell wall and its remodelling to ensure the correct form of growth (Klis et al., 2006; La Valle et al., 2000). Although no direct demonstration of enzymatic activity of Camp65p has been provided, the protein sequence analysed here has shown typical, essential motifs of the β-glucanase family of the fungal cell wall (Sestak et al., 2004). Moreover, the presence of this protein in C. albicans cell wall, its covalent binding with β-glucan and its function as a β-glucanase enzyme has recently been confirmed by a proteomic analysis of cell wall extract (de Groot et al., 2004). However, the biological role of this component has never been properly investigated.

We have used the URA3 blaster protocol to disrupt one or both CAMP65 alleles, then assessed the physiological consequences of this disruption, in terms of morphogenesis, adherence and pathogenicity of the fungus. To avoid possible bias consequent to the ectopic insertion of the URA3 gene on CAMP65 gene expression, virulence and phenotype of the fungus, we targeted reinsertion of the URA3 gene to the RPS1 locus to generate a set of isogenic wild type, heterozygous, homozygous and revertant strains. Moreover, at least two independently-generated mutants were always obtained and analysed for their genetic traits, and biological properties: they showed absolutely similar behaviour in vitro as well as in vivo.

Effective and specific gene disruption, demonstrated by Southern blotting analysis, as well as by both gene and protein expression in several controlled experiments, generated a CAMP65 null mutant which was fully viable and grew well as yeast but was severely affected in its capacity to develop hyphae on solid media. Since the heterozygous camp65/CAMP65 mutant showed only partial inability to form hyphae, a gene-dosage effect is likely, as verified with other genes involved in filamentation pathways like the HWP1 gene (Tsuchimori et al., 2000; Sharkey et al., 1999) or some pH-regulated genes coding for cell wall proteins (Saporito-Irwin et al., 1995). The specificity of the functional morphogenetic defect attributable to CAMP65 deletion was demonstrated by the rescue of full capacity of hyphae formation in the revertant heterozygous strain. The defective hyphal morphogenesis was confirmed by other experiments in liquid media, where formation of germ-tubes, i.e. the hyphae precursors, was significantly delayed or arrested in the homozygous mutant strain. Overall, the data demonstrates that CAMP65 is involved in the chain of metabolic and structural events that are necessary for correct hyphal formation.

The fact that CAMP65 is necessary for correct hyphal formation makes previous studies relating hyphal development with pathogenicity relevant (Davis et al., 2000; Tsuchimori et al., 2000; Gale et al., 1998; Leidich et al., 1998; Lo et al., 1997; Stoldt et al., 1997; Saporito-Irwin et al., 1995; Liu et al., 1994), and this is borne out by the fact that the CAMP65 disrupted mutants were clearly less pathogenic than the parental strain in a model of lethal systemic infection. However, this null mutant was also significantly less pathogenic in a model of mucosal infection, an observation that has never been reported with other hyphae-defective mutants and which further demonstrates the attractiveness of CAMP65 as a main target. The observation that the CAMP65 gene plays also a role in mucosal virulence is relevant since the virulence traits which allow mucosal or deep-seated invasion may not be the same, and the mechanisms of host response against the two infections are also different (De Bernardis et al., 1993).

As previously demonstrated and confirmed herein by a specific reaction with a monoclonal antibody, Camp65p is present on the fungal cell surface and may contribute to the adhesive properties of the fungus. In fact, the CAMP65 null mutants did not express the protein in the cell wall and were much less adhesive to plastic than the parental strains. C. albicans hyphae have pronounced adhesive properties, and it appears that the virulence of the wild type strain may be associated with Camp65p acting as an adhesin, as also suggested by the presence of an RGD signature in its sequence. The RGD motif characterizes various proteins of eukaryotic organisms involved in adhesion mechanisms, both as adhesins and as adhesin receptors (Calderone et al., 2000; Gale et al., 1998). Besides the observation made here that anti-Camp65p antibodies inhibit adherence of the fungus to plastic, we have recently obtained strong evidence that Fc-devoid, human domain antibodies against Camp65p inhibit Candida adherence to several human tissues, in addition to plastic (De Bernardis et al., 2006).

Other soluble or glucan-bound cell wall proteins (for instance, HWP1 (Tsuchimori et al., 2000; Sharkey et al., 1999) and a Candida analogue of S. cerevisiae NOT5 gene (Cheng et al., 2003)) have been implicated in adherence and/or filamentation, but this is the first report that properties such as adherence, morphogenesis and pathogenicity are directly related to a β-glucanase enzyme, the role of which was previously attributed to cell wall degradation and remodelling. By the function of this protein, hyphal cell wall construction, adherence and virulence appear to be intimately interrelated.

Besides their inherent capacity of tissue damage, hyphae of C. albicans have evolved a number of mechanisms to deviate and render host response inefficient. These include enhanced secretion of virulence enzymes degrading host proteins, antigenic variations and mimicry as well as perturbance of immune response initiated by dendritic cells (Soll, 2004; Torosantucci et al., 2004; d'Ostiani et al., 2000; Romani, 1997). Recently, the expression of Dectin-1 receptor has been demonstrated to be substantially lost on hyphal cells. Since Dectin-1 is important for phagocytosis, these observations led some authors to suggest that modulation of the above receptor is a further mechanism of immuno-evasion by C. albicans (Gantner et al., 2005). Interestingly, the dectin-1 receptor has been identified as a β-glucan molecule (Brown et al., 2003).

Other virulence traits are expressed by pathogenic strains of C. albicans, inclusive of production of hydrolases and phospholipases (Calderone and Fonzi, 2001; De Bernardis et al., 2001; Sugiyama et al., 1999; Leidich et al., 1998; Cutler, 1991). Camp65p is a mannoprotein which represents a main target of the anti-Candida immune response in humans (Torosantucci et al., 1991). Several Camp65p specific human T cell clones have been generated and shown to express a typical T-helper type 1 response (Nisini et al., 2001), generally considered as essential for anti-Candida protection (Romani, 1997).

### General Approaches and Strategies

The data described below refer to strains, mutant designation and molecular reagents for mutant construction as shown in Tables 1-3 (Examples Section, below for technical details). In the strategy of targeted CAMP65 gene disruption and re-insertion, two independently-generated mutants (i.e., two isolates for each mutant strain) were always obtained and analysed for both genetic and phenotypic traits. Since all results were totally comparable for each independently derived mutant, only the one is referred to in the following description for the sake of simplicity. To knock out CAMP65 gene, a wild type strain of C. albicans (CAI4) was initially transformed with KpnI-PstI-digested pRLV140 plasmid to release the disruption cassette CAMP65::hisG-URA3-hisG and target integration to the CAMP65 locus. After the first round of transformation, the CAMP65/camp65::hisG-URA3-hisG heterozygous strain (het1) was selected for the loss of the URA3 gene by growth on medium containing 5-FOA, and transformed again with the CAMP65::hisG-URA3-hisG disruption cassette to obtain the camp65::hisG/camp65::hisG-URA3-hisG homozygous strain (hom1). A revertant strain with CAMP65 re-insertion was also constructed (rev1, Table 1). To avoid any potential bias due to the ectopic insertion of a gene into the fungal cell (Brand et al., 2004) we, therefore, generated het2, hom2 and rev2 isogenic strains by targeted integration of URA3 at the RPS1 locus. As wild type strain, we used a wt1-derived strain, obtained by integration of URA3 at RPS1 locus (wt2; Table 1; for technical details, see also Experimental Procedures) (Murad et al., 2001). Although we noticed that the morphology, adherence to plastic and experimental pathogenicity of these isogenic strains did not substantially differ in any respect from the wt1, het1, hom1 and rev1 counterparts, the results shown here, unless otherwise stated, are those obtained with the isogenic wt2, het2, hom2 and rev2 strains. For the sake of simplicity, these strains will be hereafter designated as wild type (wt), heterozygous (het), homozygous (hom) and revertant (rev) strains, respectively.

### Targeted Disruption of the CAMP65 Gene

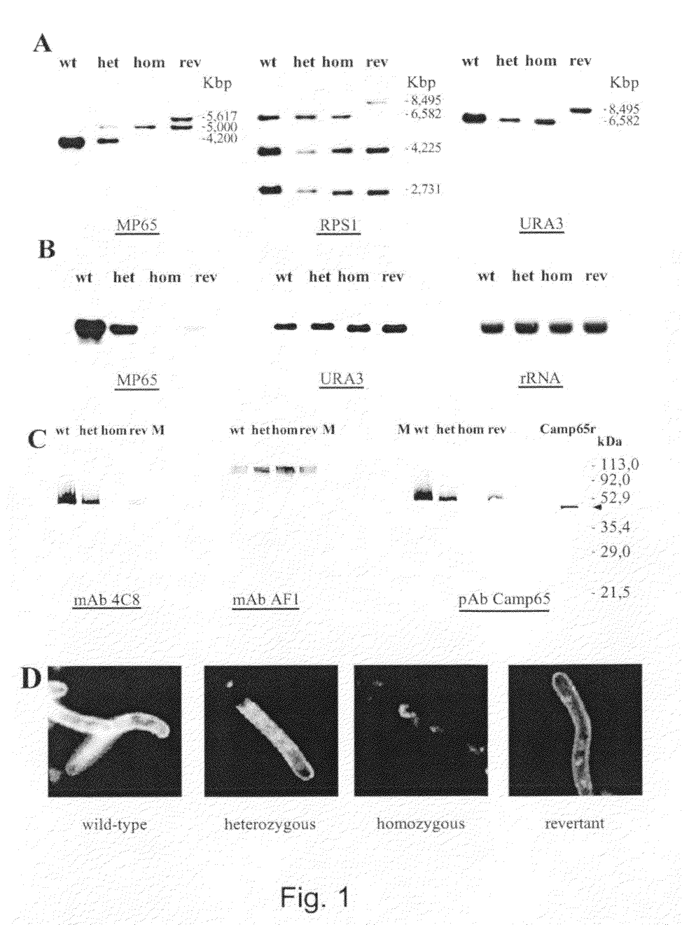

Effective and specific gene disruption and re-insertion was demonstrated by PCR (data not shown) and Southern blotting analysis (FIG. 1A). To further confirm the CAMP65 gene disruption and revertant strain construction, we performed both Northern- and Western-blot analysis with total RNA and secreted protein, respectively, purified from C. albicans cells grown in modified Lee's medium at 37° C. for 24 h. As shown in FIG. 1B, the CAMP65 gene was transcribed at a lower level in the camp65/CAMP65 heterozygous strain, and not transcribed at all in the homozygous null mutant strain which, however, expressed rRNA at a wild type level. The same pattern of CAMP65 expression was evident by testing secreted proteins of the wild type and sequential mutant strains in immuno-blotting with the anti-Camp65p polyclonal serum or the anti-Camp65 monoclonal antibody 4C8 (Gomez et al., 1996) (FIG. 1C), i.e. decrease and absence of Camp65p in heterozygous and homozygous strains, respectively.

As a further control, and because the mutant strains were endowed with different plastic adhesive properties (see below), the extract from each strain was subjected to Western blot with a monoclonal antibody (mAb AF1) (Cassone et al., 1988) recognising a mannan epitope common to different mannoprotein adhesins of C. albicans. As shown in FIG. 1C, all strains expressed polydisperse, high molecular weight mannoprotein constituents to a comparable level. Both in Northern- and Western-blot, the revertant strain recovered the ability to express CAMP65 (FIGS. 1B, 1C), thus proving the re-introduction in this strain of a functional CAMP65 gene.

Finally, immunofluorescence studies with the Camp65p-specific 4C8 mAb showed the expected bright cell wall fluorescence in the parental strain whereas the homozygous null strain did not show any cell wall fluorescence, despite the presence of some background, non-specific fluorescence in the cytoplasm. Conversely, some weak fluorescence was detected in the cell wall of both the heterozygous and revertant strains (FIG. 1D). Similar results were obtained with polyclonal anti-Camp65p serum.

It has already been reported that Camp65p from C. albicans is homologous to Scw10p from S. cerevisiae (La Valle et al., 2000), a member of the GH17 glycosyl-hydrolase (1-3-β-glucanase) family. Both of the glutamate residues Glu-326 and Glu-380 are conserved in all members of the GH17 family, being crucial for enzyme activity (Sestak et al., 2004). In order to assess whether they are present in Camp65p, we performed an amino acid alignment of barley 1,3-β-glucanase (Swiss-Prot primary accession number P15737), Bgl2p (P15703), Scw1p (Q04951) and Camp65p (Q9HEP1). Two glutamate residues in Camp65p sequence at the positions 319 and 371 were indeed found in Camp65p. This suggests that this mannoprotein is indeed a β1,3-glucanase involved in cell wall morphogenesis, as further documented below.

### CAMP65 is Required for Hyphal Morphogenesis

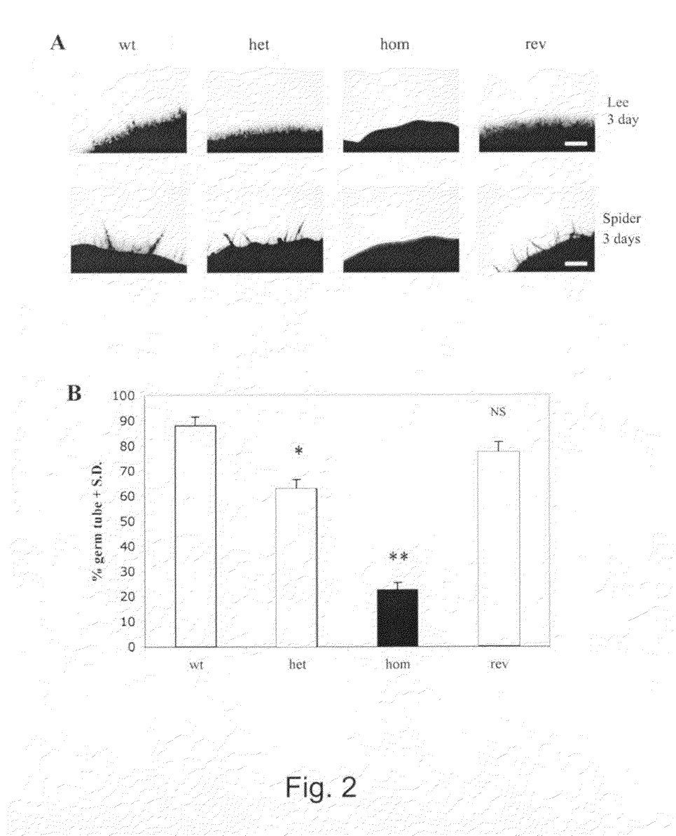

The CAMP65 gene is non-essential, as demonstrated by the full viability of null mutants (see also below). However, the mutations severely impaired hyphal differentiation. As shown in FIG. 2A, the camp65/camp65 homozygous strain showed suppressed hyphal formation when compared with the CAMP65/CAMP65 wild type strain, and this was observed on both Spider and Lee's solid media. The CAMP65/camp65 heterozygous strain showed an intermediate phenotype, suggesting a gene dosage effect. To confirm that the mutant phenotype was directly associated with CAMP65 loss, the revertant strain was grown in the same media. The ability of this strain to form hyphae was indeed restored to a level comparable to that of heterozygous strain (FIG. 2A). Wild type and mutant strains were also compared for their ability to form germ tubes, the known precursors of mature hyphae, in M199 liquid medium: the camp65/camp65 null strain was clearly impaired in germ tube formation when compared to wild type, heterozygous and revertant strains (FIG. 2B). As an additional control, we verified that the inability of the CAMP65 mutant cells to differentiate into hyphal forms was not due to a generally defective growth rate and viability. In fact, no appreciable difference in the rate of growth in YPD medium at 25° C., a temperature allowing growth under yeast form, could be observed among strains. The same experiment was repeated in Spider and Lee's media at 25° C. and concentrated (2×) YPD at 37° C. and, again, no growth differences between wild type and CAMP65 mutants were observed (data not shown). Therefore, the inability to differentiate into hyphal forms is unlikely to result from a generic growth defect.

### CAMP65 Mutants are Defective in Adherence to Plastic

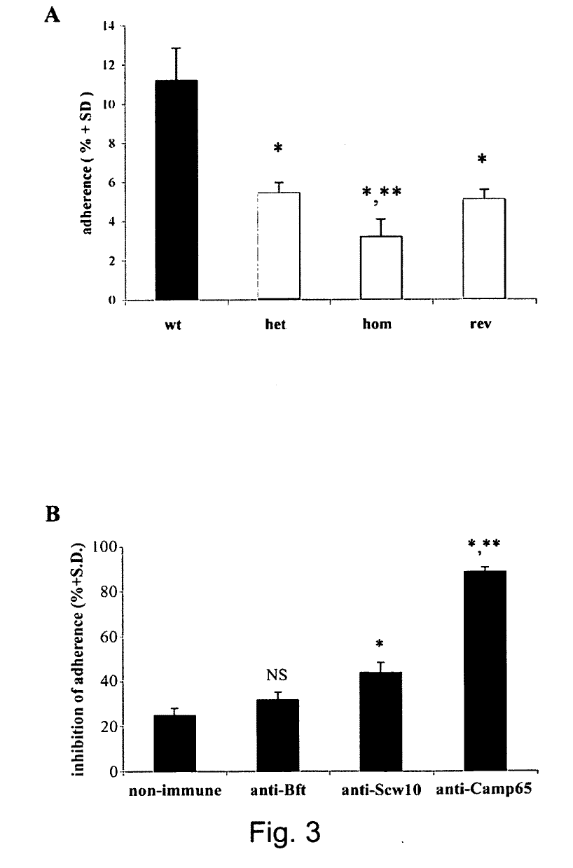

We next examined the plastic adherence properties of the CAMP65 mutants, being these properties also critical for C. albicans pathogenicity in catheter-bearing, immunocompromised subjects (Cutler, 1991). We found that deleting both copies of CAMP65 had a significant effect on fungal adherence to polystyrene plates (FIG. 3A, one typical experiment out of 3 performed with similar results). Conversely, the revertant strain adhered to the plastic to the same extent as the heterozygous strain, and both strains showed an intermediate defective phenotype compared with the wild type and homozygous cells.

Since germ-tubes and hyphal forms have increased adhesive properties with respect to the yeast cells (Cutler, 1991), we performed a series of experiments to demonstrate that the expression of CAMP65 itself, and not hyphal differentiation, was necessary for adherence. To this end, we generated an antiserum against a purified, recombinant Camp65p (La Valle et al., 2000) and a purified IgG-rich fraction of it was tested for the capacity to affect adherence to plastic of yeast-form cells of the wild type strain. In these experiments, the equivalent IgG-rich fraction of an immune serum against the Scmp65p, i.e. the Camp65p homologous protein of S. cerevisiae, (Scw10p) (La Valle et al., 2000) was also used. IgG-rich fractions of non-immune and irrelevant immune serum served as negative controls. As shown in FIG. 3B (which refers to the data of one experiment out of two performed with similar results), the anti-Camp65p serum almost totally inhibited the adherence of yeast cells to plastic. Also, the IgG-rich fraction of the serum against Scmp65p caused some inhibition, whereas an irrelevant IgG-rich serum fraction did not differ from the IgG-rich fraction of a non-immune mouse serum in causing a very low, background inhibition of adherence (this background inhibition level could be due to the presence in mouse serum of some antibodies directed against other C. albicans adhesins). Overall, these experiments demonstrate that Camp65p plays a functional role in the adherence of C. albicans cells to the plastic, whatever the form of fungus growth.

CAMP65 Mutants have Markedly Decreased Experimental Pathogenicity

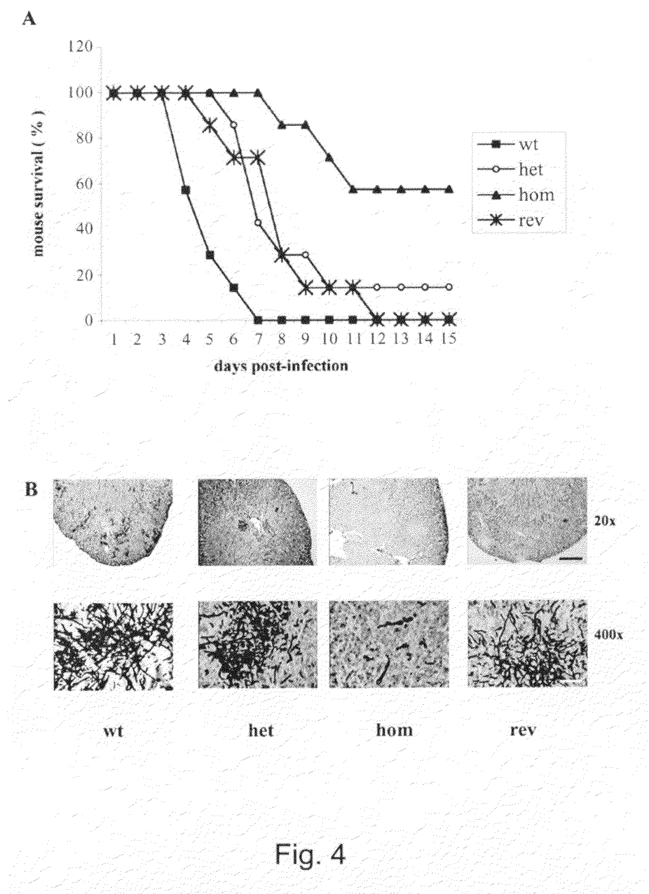

The effects of CAMP65 disruption on Candida virulence were evaluated. Thus, heterozygous, homozygous and revertant strains were first compared to the wild type strain in a lethal murine model of haematogenously disseminated candidiasis. CD2F1 mice were challenged with 5×10⁵ yeast-form cells of each strain by the intravenous route and mouse mortality was followed for up to 30 days. All mice infected with the wild type strain succumbed to candidal infection within 7 days with a median survival time of 4 days. In contrast, almost 100% of mice infected with either heterozygous or homozygous strains were alive on day 7. These results show that Camp65p-producing cells were more virulent than non-producing cells and the virulence was gene-dosage dependent. Indeed, the median survival time (days) of mice infected with the heterozygous and revertant strains was 7 and 8 days, respectively, while the MST of mice infected with homozygous strain was 17 days. In particular, about 60% of mice infected with homozygous mutant cells were still alive on day 15 (P<0.05, two tailed). FIG. 4A graphically summarizes the earliest and most significant differences in mouse survival rates. Similar comparative results were obtained by testing virulence through enumeration of colony-forming units (CFU) in kidneys of mice infected with the same strains.

Histology of the kidneys of mice infected with the wild type strain showed multiple foci of proliferating fungal hyphae together with infiltration of host inflammatory cells. On the contrary, the kidneys of mice infected by the homozygous mutant showed a few infected foci with much fewer fungal cells, associated with little or no morphological damage to the surrounding matrix. Moreover, the homozygous mutant cells presented aberrant-thicker forms that failed to form hyphae (FIG. 4B).

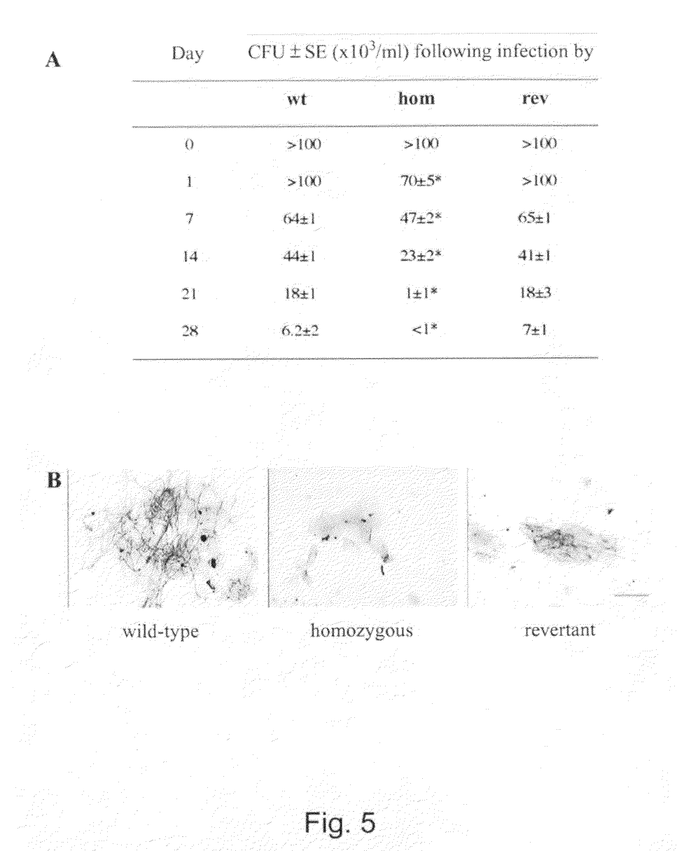

In the self-healing mucosal model of infection (De Bernardis et al., 1993), the overall kinetics of fungus clearance from rat vagina was quite similar with all mutants and wild type strain. However, the early rate of clearance (day 0-7 interval) was significantly (P<0.05, Student's t test, two tailed) more accelerated when the challenger was the homozygous strain. Moreover, all rats challenged with this strain were infection-free (less than 1 CFU/μl of vaginal fluid) on day 28, while all rats challenged with the wild type and the revertant strains were still infected (FIG. 5A). Histological observation of vaginal scrapings taken from rats infected with each individual strain clearly documented that the homozygous strain did not develop hyphae in the rat vagina at a time (2 days post-challenge) when both the wild type and the revertant strains had fully developed hyphae (FIG. 5B).

### EXAMPLES

The following examples are offered to illustrate, but not to limit the claimed invention.

### Example 1

### Microorganisms and Growth Conditions

Escherichia coli XL1 blue (endA1, hsdR17, supE44, thi1, recA1, gyrA96, relA1, Δlac, [F', proAB, lacIqZΔM15, Tn10] and M15 (nal^{S}, str^{S}, rif^{S}, lac⁻, ara⁻, gal⁻, mtl⁻, F⁻, recA⁺, uvr⁺, [pUHA1]) were used as host strains for recombinant plasmids. E. coli strains were grown in L-broth (1% tryptone, 0.5% yeast extract, 0.5% NaCl, pH 7.0) and Luria Bertani (LB) plates (1% tryptone, 0.5% yeast extract, 0.5% NaCl, 1.5% agar, pH 7.0) supplemented when necessary with ampicillin (100 μg/ml) or tetracycline (12.5 μg/ml) (La Valle et al., 1995).

Candida albicans strains used in this study are listed in Table 1. They were routinely cultured on Yeast-peptone-dextrose (YPD; 1% yeast extract, 2% bacto-peptone, 2% glucose, all w/v) or Yeast Nitrogen Base (YNB; 2% glucose, 0.17% yeast nitrogen base without amino acids and ammonium sulphate, 0.5% ammonium sulphate, w/v) or Winge (0.3% yeast extract, 0.2% glucose) or SDB (1% bacto-peptone, 2% dextrose) media at 28° C., as indicated in specific sections. All media were solidified with 2% agar and supplemented when necessary with uridine (25 μg/ml) and 5-fluoroorotic acid (5-FOA, 1 mg/ml) and chloramphenicol (50 μg/ml) (Sharkey et al., 1999).

TABLE 1
C. albicans strains described herein (all strains derived from SC5314 except ATCC20955).

| | | | | Nomenclature |
|---|---|---|---|---|
| | | | Source | used in this |
| Strain | Parent | Genotype | or ref. | study |
| SC5314 | | Clinical isolate | SC | SC5314 |
| | | | collection | |
| CAI-4 | CAF2-1 | Δura3::imm434/Δura3::imm434 | (Saporito- | — |
| | | | Irwin et al., | |
| | | | 1995) | |
| CAI12 | CAI-4 | Δura3::imm434/URA3 | (Porta et al. | wt1 |
| | | | 1999) | |
| RLVCA4 | CAI-4 | as CAI-4 but | Herein | het1 |
| | | camp65::hisG-URA3-hisG/CAMP65 | | |
| RLVCA8 | RLVCA4 | as CAI-4 but | Herein | het1 ura3 |
| | | camp65::hisG/CAMP65 | | |
| RLVCA31 | RLVCA8 | as CAI-4 but | Herein | hom1 |
| | | camp65::hisG/camp65::hisG-URA3-hisG | | |
| RLVCA35A | RLVCA31 | as CAI-4 but | Herein | hom1 ura3 |
| | | camp65::hisG/camp65::hisG | | |
| RLVCA57E | RLVCA35A | as CAI-4 but camp65:: | Herein | rev1 |
| | | hisG/camp65::hisG::CAMP65::URA3 | | |
| NGY152 | CAI-4 | as CAI-4 but | (Murad | wt2 |
| | | RPS1/rps1::CIp10 | et al., 2001) | |
| RLVCA95 | RLVCA8 | as CAI-4 but | Herein | het2 |
| | | camp65::hisG/CAMP65, | | |
| | | RPS1/rps1::CIp10 | | |
| RLVCA96 | RLVCA35A | as CAI-4 but | Herein | hom2 |
| | | camp65::hisG/camp65::hisG, | | |
| | | RPS1/rps1::CIp10 | | |
| RLVCA97 | RLVCA35A | as CAI-4 but | Herein | rev2 |
| | | camp65::hisG/camp65::hisG, | | |
| | | RPS1/rps1::CIp10 - CAMP65 | | |
| ATCC20955 | | Clinical isolate | ATCC | ATCC20955 |
| | | | collection | |

Hyphae formation (filamentation) on agar-solidified media was obtained by diluting stationary-phase cells to 2×10⁸ cells/ml in water, spotting 1×10⁶ cells onto Medium 199, Lee's, Spider and serum plates, and incubating them at 37° C. for 7 days. Solid medium 199 (M199 cat. n^{o} 31100-019, Invitrogen Corporation, Carlsbad, Calif.) was buffered with 150 mM Tris (pH 7) as previously described (Sharkey et al., 1999). Solid Spider, serum and modified Lee's medium were prepared as described previously (Liu et al., 1994).

Germ-tube formation was assessed at 37° C. in M199, modified Lee's, Spider and serum media following inoculation of stationary-phase cells into pre-warmed broth at a density of 10⁸ cells/ml. Negative controls were incubated in the same medium at 28° C. Cells were counted in a Thoma chamber.

All chemicals and antibiotics were from Roche Diagnostic (Perkin Elmer Roche, Branchburg, N.J.) and Sigma-Aldrich (Milano, Italy). Microbiological powders (except M199) were from Becton Dickinson (Becton Dickinson & Co., Sparks, Md.).

### Example 2

### Plasmid and Strain Construction

To construct the CAMP65 disruption plasmid, the 3.8 kb fragment, obtained by BamHI-Bgl II digestion of p5921 (Fonzi and Irwin, 1993), was cloned into the BclI sites of pRLV139 (La Valle et al., 2000) in order to delete a 348 bp fragment of CAMP65 (spanning from nucleotide 150 to 498 of coding sequence) and insert the hisG-URA3-hisG marker thus constructing the pRLV140 plasmid. The pRLV140 construct was then digested with KpnI and PstI, and used to transform the ura− C. albicans strain CAI4 (wild type) by a lithium acetate-based transformation protocol as described elsewhere (Fonzi and Irwin, 1993). Ura+ prototrophs were selected on YNB medium. The heterozygous CAMP65/camp65::URA3 strain RLVCA4 (het1) was plated onto 5-FOA-containing YNB plates to obtain the ura3 heterozygous RLVCA8 strain (het1 ura3). A second cycle of disruption was performed to generate URA3 homozygous CAMP65 strain RLVCA31 (hom1), which was then subjected to a second cycle of 5-FOA selection to create ura3 RLVCA35A strain (hom1 ura3).

To construct the revertant strain, we first produced the pRLV161 plasmid by amplifying CAMP65 locus with Ca72 and Ca73 oligonucleotides, as described below, and cloning into the pGEM-T vector (Promega, Madison, Wis.). The pRLV161 plasmid was digested with SacI restriction enzyme and the CAMP65 locus was cloned into the SacI site of the pSMS44 plasmid (Porta et al., 1999), to give pRLV162. The pRLV162 plasmid was then digested with BstXI restriction enzyme and the linearized plasmid was used to transform hom1 ura3 strain. Finally, URA3 revertant strain RLVCA57E (rev1) was selected on YNB.

To construct purely isogenic strains and avoid potential problems associated with the ectopic expression of URA3strains, the het1 ura3 and the hom1 ura3 strains were transformed with NotI-digested CIp10 plasmid (GenBank accession number AF181970) (Murad et al., 2000) to create the strains RLVCA95 (het2) and RLVCA96 (hom2), respectively. To construct a revertant strain, pRLV161 plasmid was digested with ApaI and SalI restriction enzymes to excise the 2087 bp fragment containing the CAMP65 locus. This was therefore cloned into the ApaI/SalI sites of the CIp10 plasmid to obtain pRLV169 plasmid. The hom1 ura3 strain was then transformed with BglII digested pRLV169 plasmid to give pRLVCA97 revertant strain (rev2). As wild type strain, we used the NGY 152 strain (wild type2, CAI-4 transformed with StuI-digested CIp10 plasmid) (Murad et al., 2000).

All recombinant plasmid inserts were fully sequenced with automated method (Genenco, Florence, Italy) by using primers flanking the poly-linker region of the vector as well as internal primers. Table 2 lists all plasmids and gene inserts used throughout this study. In the strategy of target CAMP65 gene disruption and re-insertion, at least two independently generated mutants were always obtained and analysed for both genetic and phenotypic traits. Since all results were totally comparable for each independently derived mutant, only one is referred to in the following description. Total chromosomal DNA was isolated with GFX Genomic Blood DNA Purification kit (Amersham Pharmacia Biotech Inc., Piscataway, N.J.), digested with BamHI-HindIII or BamHI to analyse CAMP65 or URA3 and RPS1 loci respectively, subjected to agarose gel electrophoresis and transferred to Hybond-N membrane (Amersham). The filters were probed with biotin-labeled DNA fragments obtained by BamHI-PvuII digestion of the pRLV130 plasmid (La Valle et al., 2000), BamHI-BglII or EcoRI-NotI digestion of Cip10 to release the 516 bp CAMP65-, 693 bp RPS1- or 586 bp URA3-fragments, respectively. The probes were labelled with North2South Biotin Random Prime Kit (Pierce, Rockford, Ill.) and purified with MicroSpin G-25 column (Amersham), while hybridisation and detection were performed with North2South Chemiluminescent Nucleic Acid Hybridisation and Detection Kit (Pierce). All enzymes were from Roche Diagnostic.

TABLE 2
Plasmids used in methods described herein.

| PLASMID | VECTOR | INSERT | Source or Ref. |
|---|---|---|---|
| pRLV130 | PDS56 (La Valle | CAMP65 cds | (La Valle |
| | et al., 1995) | | et al., 200 [symbol] |
| pRLV139 | PBLUEScript | CAMP65 cDNA | (La Valle |
| | | | et al., 200 [symbol] |
| pRLV140 | PBLUEScript | camp65::hisG- | Herein |
| | | URA3-hisG | |
| pRLV161 | pGEM-T | CAMP65 locus | Herein |
| pRLV162 | pSMS44 (Porta | CAMP65 locus | Herein |
| | et al., 1999) | | |
| pRLV169 | CIp10 (Murad | CAMP65 locus | Herein |
| | et al., 2000) | | |

[symbol]

indicates data missing or illegible when filed

### Example 3

### Polymerase Chain Reactions (PCR)

To amplify CAMP65 locus for cloning, PCR reactions were performed on a Gene Amp PCR System 9600 apparatus (Perkin Elmer Roche), in a volume of 100 μl containing 20 mM Tris-HCl pH 8.8, 10 mM KCl, 2 mM MgSO₄, 10 mM (NH₄)₂SO₄, 1% Triton X-100, 1 mg/ml nuclease-free BSA, 200 μM of each deoxynucleotide, 0.5 μM of each primer (Table 3), 5 unit of native Pfu DNA polymerase (Stratagene, La Jolla, Calif., USA) and 100 ng of fungal DNA that was purified with GFX Genomic Blood DNA Purification kit (Amersham, Pharmacia). PCR was done using the following protocol: initial denaturation at 95° C. for 45 s followed by 25 cycles of denaturation at 95° C. for 45 s, annealing at 49° C. for 45 s, extension at 72° C. for 4 (for Ca72 and Ca73) and final extension at 72° C. for 10 min. To control the CAMP65, URA3 and RPS1 loci in wild type and mutant strains, the general protocol described above was used with the following little differences: the final volume of reaction was 20 μl; PCRs were performed with 200 ng of genomic DNA as template and Taq DNA polymerase (Invitrogen); the protocol was 2 min of denaturation at 94° C. followed by 35 cycles of denaturation at 94° C. for 45 s, annealing at 50° C. for 30 s (with Cal 16/Ca117 and Cal 16/Ca119 primers to amplify URA3 and Ura3-Rps1 loci, respectively) or at 46° C. for 2 min (with Ca64/Ca65 primers to amplify CAMP65 locus), extension at 72° C. for 2 min—followed by a final extension at 72° C. for 10 min.

TABLE 3

Sequence and localisation of oligonucleotides
described herein.

| | | SEQ | |
|---|---|---|---|
| Oligo- | | ID | Local- |
| nucleotide | 5′ to 3′ sequence^{a} | NO | isation |
| Ca64 | AACGGATCCATGTTATTCAAGTCT | 1 | 2067-2084^{b} |
| | TTC | | |
| Ca65 | GGGCTGCAGGTGCTTAGTTAGAGT | 2 | 3207-3191^{b} |
| | AA | | |
| Ca72 | ATAGAGCTCGTGCGAAATTCGTCT | 3 | 1483-1501^{b} |
| | AAT | | |
| Ca73 | CTTGGTACCCGATGACTTCTAACT | 4 | 3483-3462^{b} |
| | CTTTCT | | |
| Ca116 | GCTGTAGTGCCATTGATTCGTAAC | 5 | 843-866^{c} |
| Ca117 | CTGGATCTCTTCCTTTACCAAAC | 6 | 1868-1846^{c} |
| Ca119 | CATGGAGGCCTCATGGTTTGGATT | 7 | 2573-2548^{c} |
| | TC | | |

^{a}The underlined sequences (non complementary flanking regions) contain the restriction site (in boldface) to clone the DNA fragment in the vector.
^{b}Oligonucleotide position is referred to orf 6.1672 (CAMP65 coding sequence spans from 2067 to 3203) (La Valle et al., 2000).
^{c}Oligonucleotide position is referred to CIp10 plasmid sequence (GenBank accession number AF181970) (Murad et al., 2000).

### Example 4

### Northern Blot

Total RNA from C. albicans cells grown at 37° C. for 24 h in uridine-supplemented, modified Lee's medium was isolated by RNeasy midy kit (Qiagen Hilden, Germany). Approximately 10 μg of RNA per lane was run on denaturing 1.2% formaldehyde-agarose gel, transferred to nylon membrane and probed with a biotin-labeled CAMP65 and URA3 fragments (see above) and rDNA as previously described (Sandini et al., 2002). Hybridisation and detection were performed with North2South Chemiluminescent Nucleic Acid Hybridisation and Detection Kit (Pierce).

### Example 5

### Immunoblotting

Cultures were grown at 37° C. for 24 h in 100 ml of modified Lee's medium supplemented with uridine. Supernatant from each culture was dialysed and concentrated approximately 100-fold using centrifuge filter units equipped with Biomax-5K membranes (Millipore, Bedford, Mass., USA). Concentrated samples (4 μg of proteins) were separated by SDS-PAGE on 10% polyacrylamide gels and then electro-transferred to nitrocellulose membranes (Biorad, Hercules, Calif., USA) as previously described (Sandini et al., 1999). Non-specific binding of antibodies to nitrocellulose was prevented by blocking the filters with 3% bovine serum albumin in Tris-buffered saline (TBS) for 2 h at 37° C. After three washes (10′ each) with TBS, 0.05% Tween20 (TBST), membranes were incubated with mouse anti-Camp 65p polyclonal serum (La Valle et al., 2000) (diluted 1:5.000 in TBS, BSA 3%) or anti-mannan monoclonal antibody (mAb) AF1 (Cassone et al., 1988) (diluted 1:20 in TBS, BSA 3%), overnight at 4° C. and reacted with alkaline phosphatase (AP)-conjugate goat anti-mouse IgG (1:15.000) (Sigma) or with AP-conjugate goat anti-mouse IgM (1:30.000) (Sigma), for 2 h at room temperature (RT), respectively. After rinsing three times with TBST, the membranes were placed in BCIP/NBT visualisation solution (Ausubel F M, 1996) and the reaction was stopped in distilled water.

### Example 5

### Growth Curves

50 ml of YPD medium (1× or 2×), Spider and Lee's media supplemented with uridine were inoculated with an overnight culture of each strain (wild type and MP65-mutants strains) at 0.1 initial optical density (OD₆₀₀) and were shaken in an orbital incubator at 28° C. or 37° C. Growth was measured spectrophotometrically at 600 nm (Hube et al., 1997; Lo et al., 1997; Sanglard et al., 1997).

### Example 6

### Immunofluorescence Assay

The wild type and CAMP65 mutants cells were grown overnight in Winge medium at 28° C., washed twice with water, resuspended at 10⁷ c/ml in LEE+U and grown at 37° C. for 2 h for hyphae induction. The cells were washed twice with PBS and resuspended in half volume in the same buffer. Then, 301 of each cell suspension were spotted onto the wells (each sample in duplicate) and dried. The cells were incubated with the anti-Camp65p mAb 4C8 (1:10) for 1 h in ice. After three washes with cold PBS, the cells were incubated in ice for 1 h in the dark, with FITC-conjugate goat anti-mouse IgG (1:64) (SIGMA). After three washes, the samples were examined under the fluorescence microscope. Negative controls consisted of cells stained in presence of a mAb with irrelevant specificity (directed against Bacteroides fragilis) and then incubated with FITC-labeled secondary antibody or cells stained with FITC-labeled secondary antibody alone.

### Example 7

### Microscopy and Imaging

The patches and cells were imaged through the agar containing plastic petri dish and slides respectively with Nikon EclipseE800 (Nikon Corporation, Tokio, Japan). Nikon Coolpix 995 was used to capture images (Nikon Corporation). Images were imported in Adobe Photoshop 7 (Adobe System Incorporated, San Jose, Calif.), converted to grayscale, enhanced in contrast and filtered to remove noise. The size of the final image was reduced and then cropped images were assembled into figures using Canvas 9 (Deneba, Miami, Fla.).

### Example 8

### Adherence Assay

Fungal cells were grown for 24 h at 28° C. in YNB broth (0.5% glucose, 0.67% YNB dehydrated), washed twice with water and suspended at 1.5×10³ cells/ml in modified Lee's or M199 liquid media. 1.5×10³ cells were incubated for 3 h at 37° C. in 6-well polystyrene plates (Corning Incorporated, Corning, N.Y., USA). After extensive washing, 1 ml of Sabouraud dextrose agar was poured in each well and allowed to solidify. After incubation at 37° C. for 24 h, colonies were counted and the results expressed as percentage of the inoculum. The inoculum size of each cell suspension was confirmed by plating aliquots of the culture directly in Sabouraud dextrose agar plates. To test the inhibitory effects of antibodies on adherence, the cells were washed three times in 0.85% NaCl, then pre-incubated for 2 h at 37° C. with 50 μg of IgG-rich fractions separated (Melon Gel IgG Spin purification kit; Pierce) from non-immune, anti-Camp65p (La Valle et al., 2000), anti-Bft1p (the B. fragilis toxin)(Sandini et al., 2001) and anti-Scw10p (the S. cerevisiae homologous of Camp65p;)(La Valle et al., 2000) hyperimmune mouse antisera. The incubation mixtures were therefore transferred to polystirene wells, allowed to adhere to plastic for 3 h at 37° C., and adherence measured as reported above.

### Example 9

### Systemic Infection of Mice

Strains of C. albicans were pre-grown overnight in Winge, streaked onto SDA plates supplemented with chloramphenicol (50 μg/ml) and incubated for two days at 28° C. Cells were harvested, washed, counted and suspended to a density of 5×106 cells/ml in sterile phosphate-buffered saline (PBS) and ten CD2F1 female mice (18-21 g, Charles River Laboratories, Wilmington, Mass.) per each C. albicans strain, were administered intravenously 0.1 ml of the suspension. Seven mice of each group were observed daily for 30 days to monitor the survival. Three mice of each group were sacrificed three days post-infection, the left and right kidneys were removed and used for colony forming unit (CFU) determination and histopathological observations, respectively. The left kidneys were homogenized in 10 ml of saline and serial dilutions plated on SDA supplemented with chloramphenicol (see above). The plates were incubated at 37° C. for 48 h, after which CFU was determined. Values were expressed as log CFU/kidney of tissue homogenized. Colonies were then streaked onto CHROMagar Candida plates to confirm identification.

For histological observation, the right kidneys were removed from mice and immediately fixed in 10% (vol/vol) neutral buffered formalin. After de-hydration in ethanol, clearing with Noxil (Italscientifica, Italia), and paraffin embedding, 6 μm-thick sections were stained with Grocott stain (BioOptica) and observed under a light microscope. The images were captured with Nikon Microphot-Fx and Arkon software at different magnifications, imported in Adobe Photoshop 7 and then assembled into figures using Canvas 9.

### Example 10

### Rat Vaginal Infection

Oophorectomized female Wistar rats (80-100 g, Charles River Breeding Laboratories, Calco, Italy) were used throughout this study. Animal maintenance and overall care were as described elsewhere (De Bernardis et al., 1999; Ghannoum and Abu Elteen, 1986). All rats were maintained under pseudoestrus by injection of estradiol benzoate (Amsa Farmaceutici srl, Rome, Italy). Six days after the first estradiol dose, the animals were inoculated intravaginally with 10⁷ yeast cells in 0.1 ml of saline solution. The number of cells in the vaginal fluid was counted by culturing 10 μl samples (using calibrated plastic loop, Disponoic, PBI, Milan, Italy) taken from each animal, on Sabouraud agar plate containing chloramphenicol (50 μg/ml) as previously described (De Bernardis et al., 1999; Ghannoum and Abu Elteen, 1986). The rat was considered infected when at least 1 CFU was present in the vaginal lavage, i.e. a count of ≧10³ CFU/ml. Other vaginal samples were also stained by periodic acid-Schiff-van Gieson method for microscopic examination.

### Example 11

### Statistics

Quantitative data were assessed by both parametric and non-parametric statistics, as requested, and indicated in specific experiments. The significance was set at P<0.05, two tailed.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, many of the techniques and apparatus described above can be used in various combinations.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

### REFERENCES CITED HEREIN

Ausubel F M, B. R., Kigston R E, Moore D D, Seidam J G, Smith J A, Struhl K (1996) Current protocols in molecular biology. Chichester, UK.
Brand, A., MacCallum, D. M., Brown, A. J., Gow, N. A. and Odds, F. C. (2004) Ectopic expression of URA3 can influence the virulence phenotypes and proteome of Candida albicans but can be overcome by targeted reintegration of URA3 at the RPS10 locus. Eukaryot Cell. 3: 900-909.
Bromuro, C., Torosantucci, A., Gomez, M. J., Urbani, F. and Cassone, A. (1994) Differential release of an immunodominant 65 kDa mannoprotein antigen from yeast and mycelial forms of Candida albicans. J Med Vet Mycol. 32: 447-459.
Bromuro, C., Torosantucci, A., Chiani, P., Conti, S., Polonelli, L. and Cassone, A. (2002) Interplay between protective and inhibitory antibodies dictates the outcome of experimentally disseminated Candidiasis in recipients of a Candida albicans vaccine. Infect Immun. 70: 5462-5470.
Bromuro, C., La Valle, R., Sandini, S., Urbani, F., Ausiello, C. M., Morelli, L., et al (1998) A 70-kilodalton recombinant heat shock protein of Candida albicans is highly immunogenic and enhances systemic murine candidiasis. Infect Immun. 66: 2154-2162.
Brown, G. D., Herre, J., Williams, D. L., Willment, J. A., Marshall, A. S, and Gordon, S. (2003) Dectin-1 mediates the biological effects of beta-glucans. J Exp Med. 197: 1119-1124.
Calderone, R., Suzuki, S., Cannon, R., Cho, T., Boyd, D., Calera, J., et al (2000) Candida albicans: adherence, signaling and virulence. Med Mycol. 38 Suppl 1: 125-137.
Calderone, R. A. and Fonzi, W. A. (2001) Virulence factors of Candida albicans. Trends Microbiol. 9: 327-335.
Cassone, A. (1989) Cell wall of Candida albicans: its functions and its impact on the host. Curr Top Med Mycol. 3: 248-314.
Cassone, A., Torosantucci, A., Boccanera, M., Pellegrini, G., Palma, C. and Malavasi, F. (1988) Production and characterisation of a monoclonal antibody to a cell-surface, glucomannoprotein constituent of Candida albicans and other pathogenic Candida species. J Med Microbiol. 27: 233-238.
Cassone, A., De Bernardis, F., Ausiello, C. M., Gomez, M. J., Boccanera, M., La Valle, R. and Torosantucci, A. (1998) Immunogenic and protective Candida albicans constituents. Res Immunol. 149: 289-299; discussion 504.
Cheng, S., Clancy, C. J., Checkley, M. A., Handfield, M., Hillman, J. D., Progulske-Fox, A., et al (2003) Identification of Candida albicans genes induced during thrush offers insight into pathogenesis. Mol Microbiol. 48: 1275-1288.
Cutler, J. E. (1991) Putative virulence factors of Candida albicans. Annu Rev Microbiol. 45: 187-218.
d'Ostiani, C. F., Del Sero, G., Bacci, A., Montagnoli, C., Spreca, A., Mencacci, A., et al (2000) Dendritic cells discriminate between yeasts and hyphae of the fungus Candida albicans. Implications for initiation of T helper cell immunity in vitro and in vivo. J Exp Med. 191: 1661-1674.
Davis, D., Edwards, J. E., Jr., Mitchell, A. P. and Ibrahim, A. S. (2000) Candida albicans RIM101 pH response pathway is required for host-pathogen interactions. Infect Immun. 68: 5953-5959.
De Bernardis, F., Lorenzini, R. and Cassone, A. (1999) Rat model of Candida vaginal infection. In Handbook of Animal models of infection. Zack, O., and Sande, M. (eds.) London: Academic Press Ltd., pp. 735-740.
De Bernardis, F., Sullivan, P. A. and Cassone, A. (2001) Aspartyl proteinases of Candida albicans and their role in pathogenicity. Med Mycol. 39: 303-313.
De Bernardis, F., Adriani, D., Lorenzini, R., Pontieri, E., Carruba, G. and Cassone, A. (1993) Filamentous growth and elevated vaginopathic potential of a nongerminative variant of Candida albicans expressing low virulence in systemic infection. Infect Immun. 61: 1500-1508.
De Bernardis, F., Liu, H., O'Mahony, R., La Valle, R., Bartollino, S., Sandini, S., et al (2006) submitted.
de Groot, P. W., de Boer, A. D., Cunningham, J., Dekker, H. L., de Jong, L., Hellingwerf, K. J., et al (2004) Proteomic analysis of Candida albicans cell walls reveals covalently bound carbohydrate-active enzymes and adhesins. Eukaryot Cell. 3: 955-965.
Fidel, P. L., Jr. and Sobel, J. D. (1996) Immunopathogenesis of recurrent vulvovaginal candidiasis. Clin Microbiol Rev. 9: 335-348.
Fonzi, W. A. and Irwin, M. Y. (1993) Isogenic strain construction and gene mapping in Candida albicans. Genetics. 134: 717-728.
Gale, C. A., Bendel, C. M., McClellan, M., Hauser, M., Becker, J. M., Berman, J. and Hostetter, M. K. (1998) Linkage of adhesion, filamentous growth, and virulence in Candida albicans to a single gene, INT1. Science. 279: 1355-1358.
Gantner, B. N., Simmons, R. M. and Underhill, D. M. (2005) Dectin-1 mediates macrophage recognition of Candida albicans yeast but not filaments. Embo J. 24: 1277-1286.
Ghannoum, M. and Abu Elteen, K. (1986) Correlative relationship between proteinase production, adherence and pathogenicity of various strains of Candida albicans. J Med Vet Mycol. 24: 407-413.
Gomez, M. J., Torosantucci, A., Arancia, S., Maras, B., Parisi, L. and Cassone, A. (1996) Purification and biochemical characterization of a 65-kilodalton mannoprotein (MP65), a main target of anti-Candida cell-mediated immune responses in humans. Infect Immun. 64: 2577-2584.
Gomez, M. J., Maras, B., Barca, A., La Valle, R., Barra, D. and Cassone, A. (2000) Biochemical and immunological characterization of MP65, a major mannoprotein antigen of the opportunistic human pathogen Candida albicans. Infect Immun. 68: 694-701.
Hube, B., Sanglard, D., Odds, F. C., Hess, D., Monod, M., Schafer, W., et al (1997) Disruption of each of the secreted aspartyl proteinase genes SAP1, SAP2, and SAP3 of Candida albicans attenuates virulence. Infect Immun. 65: 3529-3538.
Klis, F. M., Boorsma, A. and De Groot, P. W. (2006) Cell wall construction in Saccharomyces cerevisiae. Yeast. 23: 185-202.
La Valle, R., Bromuro, C., Ranucci, L., Muller, H. M., Crisanti, A. and Cassone, A. (1995) Molecular cloning and expression of a 70-kilodalton heat shock protein of Candida albicans. Infect Immun. 63: 4039-4045.
La Valle, R., Sandini, S., Gomez, M. J., Mondello, F., Romagnoli, G., Nisini, R. and Cassone, A. (2000) Generation of a recombinant 65-kilodalton mannoprotein, a major antigen target of cell-mediated immune response to Candida albicans. Infect Immun. 68: 6777-6784.
Leidich, S. D., Ibrahim, A. S., Fu, Y., Koul, A., Jessup, C., Vitullo, J., et al (1998) Cloning and disruption of caPLB1, a phospholipase B gene involved in the pathogenicity of Candida albicans. J Biol Chem. 273: 26078-26086.
Liu, H., Kohler, J. and Fink, G. R. (1994) Suppression of hyphal formation in Canidida albicans by mutation of a STE12 homolog. Science. 266: 1723-1726.
Lo, H. J., Kohler, J. R., DiDomenico, B., Loebenberg, D., Cacciapuoti, A. and Fink, G. R. (1997) Nonfilamentous C. albicans mutants are avirulent. Cell. 90: 939-949.
Mansour, M. K. and Levitz, S. M. (2003) Fungal Mannoproteins: the sweet path to immunodominance. ASM news. 69: 595-600.
Mencacci, A., Torosantucci, A., Spaccapelo, R., Romani, L., Bistoni, F. and Cassone, A. (1994) A mannoprotein constituent of Candida albicans that elicits different levels of delayed-type hypersensitivity, cytokine production, and anticandidal protection in mice. Infect Immun. 62: 5353-5360.
Murad, A. M., Lee, P. R., Broadbent, I. D., Barelle, C. J. and Brown, A. J. (2000) CIp10, an efficient and convenient integrating vector for Candida albicans. Yeast. 16: 325-327.
Murad, A. M., Leng, P., Straffon, M., Wishart, J., Macaskill, S., MacCallum, D., et al (2001) NRG1 represses yeast-hypha morphogenesis and hypha-specific gene expression in Candida albicans. Embo J. 20: 4742-4752.
Nisini, R., Romagnoli, G., Gomez, M. J., La Valle, R., Torosantucci, A., Mariotti, S., et al (2001) Antigenic properties and processing requirements of 65-kilodalton mannoprotein, a major antigen target of anti-Candida human T-cell response, as disclosed by specific human T-cell clones. Infect Immun. 69: 3728-3736.
Pfaller, M. A., Jones, R. N., Doern, G. V., Sader, H. S., Hollis, R. J. and Messer, S. A. (1998) International surveillance of bloodstream infections due to Candida species: frequency of occurrence and antifungal susceptibilities of isolates collected in 1997 in the United States, Canada, and South America for the SENTRY Program. The SENTRY Participant Group. J Clin Microbiol. 36: 1886-1889.
Porta, A., Ramon, A. M. and Fonzi, W. A. (1999) PRR1, a homolog of Aspergillus nidulans palF, controls pH-dependent gene expression and filamentation in Candida albicans. J Bacteriol. 181: 7516-7523.
Romani, L. (1997) The T cell response against fungal infections. Curr Opin Immunol. 9: 484-490.
Sandini, S., d'Abusco, A. S., La Valle, R. and Pantosti, A. (2001) Production of a mouse antiserum to Bacteroides fragilis enterotoxin using a recombinant enterotoxin precursor. Clin Diagn Lab Immunol. 8: 190-191.
Sandini, S., Melchionna, R., Bromuro, C. and La Valle, R. (2002) Gene expression of 70 kDa heat shock protein of Candida albicans: transcriptional activation and response to heat shock. Med Mycol. 40: 471-478.
Sandini, S., Melchionna, R., Arancia, S., Gomez, M. J. and La Valle, R. (1999) Generation of a highly immunogenic recombinant enolase of the human opportunistic pathogen Candida albicans. Biotechnol Appl Biochem. 29 (Pt 3): 223-227.
Sanglard, D., Hube, B., Monod, M., Odds, F. C. and Gow, N. A. (1997) A triple deletion of the secreted aspartyl proteinase genes SAP4, SAP5, and SAP6 of Candida albicans causes attenuated virulence. Infect Immun. 65: 3539-3546.
Saporito-Irwin, S. M., Birse, C. E., Sypherd, P. S, and Fonzi, W. A. (1995) PHR1, a pH-regulated gene of Candida albicans, is required for morphogenesis. Mol Cell Biol. 15: 601-613.
Sestak, S., Hagen, I., Tanner, W. and Strahl, S. (2004) Scw10p, a cell-wall glucanase/transglucosidase important for cell-wall stability in Saccharomyces cerevisiae. Microbiology. 150: 3197-3208.
Sharkey, L. L., McNemar, M. D., Saporito-Irwin, S. M., Sypherd, P. S, and Fonzi, W. A. (1999) HWP1 functions in the morphological development of Candida albicans downstream of EFG1, TUP1, and RBF1. J Bacteriol. 181: 5273-5279.
Soll, D. R. (2004) Mating-type locus homozygosis, phenotypic switching and mating: a unique sequence of dependencies in Candida albicans. Bioessays. 26: 10-20.
Stoldt, V. R., Sonneborn, A., Leuker, C. E. and Ernst, J. F. (1997) Efg1p, an essential regulator of morphogenesis of the human pathogen Candida albicans, is a member of a conserved class of bHLH proteins regulating morphogenetic processes in fungi. Embo J. 16: 1982-1991.
Sugiyama, Y., Nakashima, S., Mirbod, F., Kanoh, H., Kitajima, Y., Ghannoum, M. A. and Nozawa, Y. (1999) Molecular cloning of a second phospholipase B gene, caPLB2 from Candida albicans. Med Mycol. 37: 61-67.
Torosantucci, A., Gomez, M. J., Bromuro, C., Casalinuovo, I. and Cassone, A. (1991) Biochemical and antigenic characterization of mannoprotein constituents released from yeast and mycelial forms of Candida albicans. J Med Vet Mycol. 29: 361-372.
Torosantucci, A., Romagnoli, G., Chiani, P., Stringaro, A., Crateri, P., Mariotti, S., et al (2004) Candida albicans yeast and germ tube forms interfere differently with human monocyte differentiation into dendritic cells: a novel dimorphism-dependent mechanism to escape the host's immune response. Infect Immun. 72: 833-843.
Tsuchimori, N., Sharkey, L. L., Fonzi, W. A., French, S. W., Edwards, J. E., Jr. and Filler, S. G. (2000) Reduced virulence of HWP1-deficient mutants of Candida albicans and their interactions with host cells. Infect Immun. 68: 1997-2002.
Yuan, R., Casadevall, A., Spira, G. and Scharff, M. D. (1995) Isotype switching from IgG3 to IgG1 converts a nonprotective murine antibody to Cryptococcus neoformans into a protective antibody. J Immunol. 154: 1810-1816.

What is claimed is:

1. A composition comprising an antibody specific for CAMP65 in a formulation for topical administration.

2. The composition of claim 1, wherein the formulation is selected from the group consisting of: suppositories, creams, pastes, ointments and gels.

3. A method of preventing adhesion of a fungus to a surface, the method comprising:
contacting a Candida spp. with an antibody-like molecule specific for CAMP65 in an amount sufficient to reduce adhesion of the Candida spp. to the surface as compared to adhesion of the Candida spp. without said contacting.

4. The method of claim 3, wherein the Candida spp. is Candida albicans.

5. The method of claim 3, wherein the surface is selected from the group consisting of: a polymer, a cell surface, a tissue surface, an organ surface, and the external surface of an animal.

6. A method of treatment for a fungal infection comprising the administration of an effective amount of an antibody-like molecule specific for CAMP65p of Candida albicans to a patient in need thereof.

7. The method of claim 6, wherein the antibody-like molecule is selected from the group consisting of: human antibodies, animal antibodies, humanized antibodies, antibody fragments, engineered fragments of antibodies, and substances incorporating a domain or region corresponding to that of a variable region of an antibody specific for CAMP65.

8. The method of claim 6, wherein said infecting fungus is selected from Candida spp.

9. The method of claim 6, wherein the infecting fungus is C. albicans.

10. The method of claim 6, wherein the medicament is prepared as an external formulation.

11. The method of claim 6, wherein the medicament is in a form selected from the group consisting of: pessaries, suppositories, flushes, creams, pastes, ointments, and gels.

12. The method of claim 6, wherein the medicament further comprises an anti-fungal agent.

13. The method of claim 6, wherein the medicament comprises at least one further antibody-like molecule to another determinant of said fungus to be treated.

14. A medicament comprising one or more antibody-like molecules specific for CAMP65p of Candida albicans.

15. An antibody-like molecule specific for CAMP65p of Candida albicans.

16. The antibody-like molecule of claim 15, wherein the antibody-like molecule is selected from the group consisting of: human antibodies, animal antibodies, humanized antibodies, antibody fragments, engineered fragments of antibodies, and substances incorporating a domain or region corresponding to that of a variable region of an antibody specific for CAMP65.

17. A vaccine for candidiasis comprising all or part of CAMP65 in an antigenic form.

18. The vaccine of claim 17, comprising a further antigenic determinant of C. albicans.
